(19) United States
(12) Patent Application Publication
(10) Pub. No.: US 2013/0172798 A1
(43) Pub. Date: Jul. 4, 2013

(54) ORTHOTIC DEVICE HAVING RELEASABLY ATTACHED PANEL EXTENSIONS AND METHOD FOR USING THE SAME

(71) Applicants: Ed Bannister, Riverside, CA (US); Michael L. Martin, Bainbridge Island, WA (US); Sai Chung Chan, Irvine, CA (US)

(72) Inventors: Ed Bannister, Riverside, CA (US); Michael L. Martin, Bainbridge Island, WA (US); Sai Chung Chan, Irvine, CA (US)

(73) Assignee: Bio Cybernetics International, Inc.

(21) Appl. No.: 13/358,262

(22) Filed: Jan. 25, 2012

Related U.S. Application Data
(60) Provisional application No. 61/581,218, filed on Dec. 29, 2011.

(51) Int. Cl. A61F 5/00

(52) U.S. Cl. 602/19

(57) ABSTRACT

Described herein is an orthotic device and a method for using an orthotic device to provide a variable range of body support. The orthotic device can comprise a body brace, a primary body panel, and a secondary body panel configured to be releasably attached to the primary body panel. The subject invention addresses the problem of patients being required to purchase multiple orthotic devices in order to receive multiple levels of support by allowing a single orthotic device to provide a variable amount of support. In one embodiment, the variable amount of support is achieved by providing both a primary body panel and a secondary body panel, with the secondary body panel configured to be removably attached to the primary body panel.

### CROSS-REFERENCE TO RELATED APPLICATION

The present application claims priority to U.S. Provision Application No. 61/581,218, entitled “Orthotic Device Having Releasably Attached Panel Extensions and Method for Using the Same,” filed Dec. 29, 2011, the entire contents of which are herein incorporated by reference.

### BACKGROUND OF THE INVENTION

The present invention relates generally to orthotic devices and methods for using an orthotic device, and more particularly to a body brace for medical or recuperative purposes having the ability to provide a variable amount of support.

An orthotic device or orthosis (commonly known as a brace or splint) is an orthopedic device that is typically applied to a limb or the body. Among other things, the purpose can be to provide support, protection, pain reduction, or replacement of lost function.

In this regard, a common method of alleviating pain in people suffering from back pain or injuries and promoting healing in post-operative back surgery patients is to stabilize the spine by means of an orthosis, such as a brace. There are a large variety of braces available depending on the diagnosis and physical needs of the individual. These devices include a multitude of construction materials and designs which can be snugly fitted around the patient's trunk and peripheral area such as the cervical and pelvic regions.

Such braces are effective in achieving spinal stability if worn properly and consistently. As a patient's condition changes, it is sometimes desirable for the patient to switch to a brace that provides either more support for a worsening condition, or less support and greater comfort and flexibility for an improving condition. For patients with improving conditions, switching braces also permits a gradual reduction in back support so that the patient may slowly be weaned off of use of the brace.

Prior art back braces provide a single level of support. To receive different levels of support, a patient must purchase multiple back braces. However, under current Medicare policies, a patient may receive a Medicare reimbursement for only one back brace. Thus, if a patient with an improving or worsening back condition wishes to switch to a back brace that provides less or more support, the patient must purchase a separate back brace using his or her own money with no reimbursement by Medicare.

Prior art back braces also have a primary body panel that may be removably attached to a rear portion of a body brace. It has been found that the prior art back braces can require an undesired amount of effort for attaching and detaching the primary body panel.

It can readily be appreciated that there is a need for a brace that can provide multiple levels of support as a patient's condition changes so that the patient does not have to purchase multiple braces, at the patient's own expense, in order to receive multiple levels of support. It can further be appreciated that there is a need for a brace comprising a primary body panel having attachment means that require less effort than in the prior art. The present invention fulfills these needs and provides further related advantages.

### SUMMARY OF THE INVENTION

The present invention is embodied in an orthotic device comprising a brace and body panels that provide support to certain parts of the human body. In one embodiment, the subject invention addresses the problem of a patient having to purchase multiple orthotic devices in order to receive multiple levels of support, by allowing a single orthotic device to provide a variable amount of support.

In one embodiment, the orthotic device comprises a body brace configured to be worn around a portion of a human body, a primary body panel, and a secondary body panel. The primary body panel is positioned on a rear portion of the body brace, and the secondary body panel is configured to be releasably attached to the primary body panel for providing an adjustable level of support to the portion of the human body.

In another embodiment, the primary body panel has hook-and-loop fastener material and the secondary body panel has complementary hook-and-loop fastener material, such that when the hook-and-loop fastener materials come into contact, the secondary body panel is releasably attached to the primary body panel.

In a further embodiment, the primary body panel and the secondary body panel are configured in such a way that the secondary body panel can be releasably attached to the primary body panel in a plurality of positions.

In another embodiment, the body brace includes two body brace segments, a pulley system mounted on the two body brace segments, and a cord wrapped around the pulley system. By adjusting the tension in the cord, the user is able to cause the pulley system to pull the body brace segments closer together or further apart, which adjusts the tightness of the body brace on the user.

The present invention is also embodied in a method for using an orthotic device to receive adjustable levels of body support. In one embodiment, the method comprises the steps of obtaining an orthotic device as described above, wearing the orthotic device with the secondary body panel attached to the primary body panel, doffing the orthotic device, removing the secondary body panel from the primary body panel, and wearing the orthotic device without the secondary body panel attached to the primary body panel. The method may further comprise the step of adjusting the body brace by pulling a cord.

The present invention is also embodied in an orthotic device comprising a body brace configured to be worn around a portion of a human body and a body panel positioned on a rear portion of the body brace. In one embodiment, the body panel comprises a plurality of apertures and a plurality of retaining posts for removably attaching the body panel to the rear portion of the body brace. Each of the apertures extends about one of the retaining posts.

In one embodiment, the plurality of apertures comprises an upper set of two apertures and a lower set of two apertures. Each of the apertures comprises two slotted portions for receiving a strap. Each of the retaining posts comprises a flanged bottom portion. The flanged bottom portion comprises two prongs extending from opposite ends of the flanged bottom portion.

These and other features and advantages of the invention should become more readily apparent from the detailed description of the preferred embodiments set forth below taken in conjunction with the accompanying drawings, which illustrate, by way of example, the principles of the invention.

### BRIEF DESCRIPTION OF THE DRAWINGS

Embodiments of the present invention will now be described, by way of example only, with reference to the following drawings.

FIG. 1 is a perspective view of an orthotic device having a removably attached secondary body panel and being worn by a person, in accordance with an embodiment of the present invention.

FIG. 2 is a perspective view of the orthotic device of FIG. 1 being worn by a person but with the secondary body panel removed.

FIG. 3 is a front elevation view of the orthotic device of FIG. 1 with two secondary body panels removably attached.

FIG. 4 is a front elevation view of the orthotic device of FIG. 1 with the secondary body panels removed.

FIG. 5 is a rear elevation view of the orthotic device of FIG. 1 with two secondary body panels removably attached.

FIG. 6 is a rear elevation view of the orthotic device of FIG. 1 with the secondary body panels removed.

FIG. 7 is a front elevation view of a left secondary body panel in accordance with an embodiment of the present invention.

FIG. 8 is a rear elevation view of the left secondary body panel of FIG. 7.

FIG. 9. is a rear elevation view of an orthotic device having a primary body panel positioned on a rear portion of the device, in accordance with an embodiment of the present invention.

FIG. 10 is a rear elevation view of a primary body panel for positioning on a rear portion of an orthotic device, in accordance with an embodiment of the present invention.

### DETAILED DESCRIPTION OF THE PREFERRED EMBODIMENTS

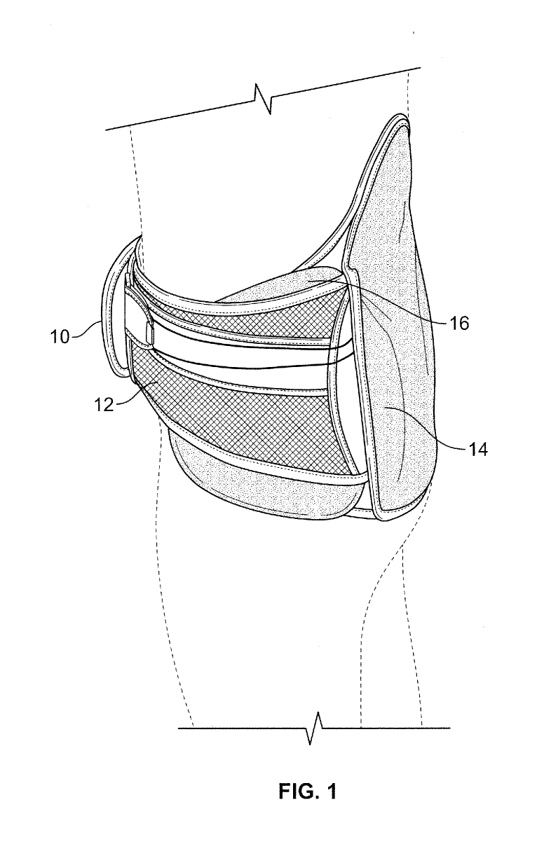

With reference to FIG. 1, there is shown an orthotic device 10 in accordance with an embodiment of the present invention. The orthotic device 10 comprises a body brace 12 that is configured to be wrapped around the torso of a user, a primary body panel 14 that is positioned on a rear portion of the body brace 12, and a secondary body panel 16 that is configured to be releasably attached to the left side of the primary body panel 14 for providing an adjustable level of support to a portion of the user's body. FIG. 1 shows a left secondary body panel. A corresponding right secondary body panel may be releasably attached to the right side of the primary body panel for providing an adjustable level of support to the right side of the user's body.

In this embodiment, the portion of the user's body receiving support is the user's torso and back. FIG. 1 shows the user wearing the orthotic device 10 with the secondary body panel 16 attached to the primary body panel 14 so as to provide the user with additional support. The secondary body panel is removably attached to the primary body panel, and the user may wear the orthotic device with the secondary body panel removed, so as to receive less support while gaining greater comfort and flexibility.

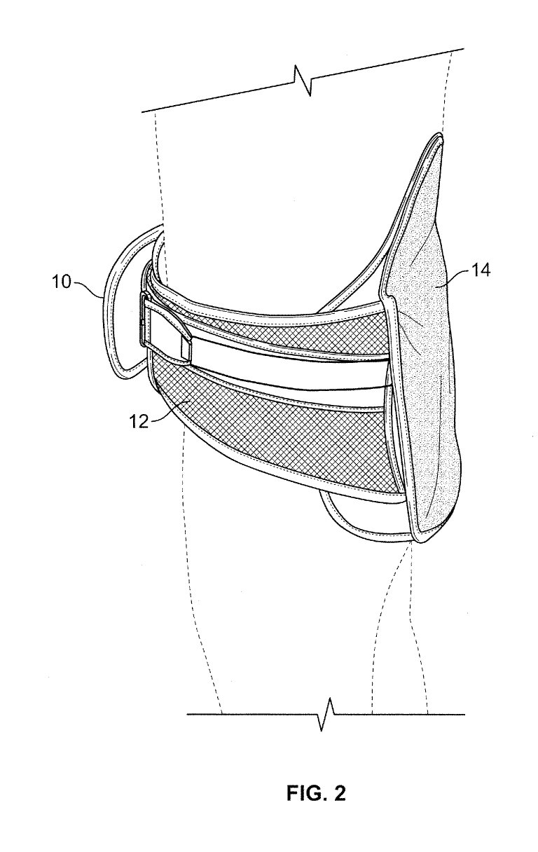

FIG. 2 depicts the orthotic device 10 with the secondary body panel 16 removed, leaving only the primary body panel 14 to provide support to the user's back. The orthotic device still comprises the body brace 12 and the primary body panel, but the secondary body panel 16 is no longer attached and no longer provides support to the user. This configuration gives the user greater comfort and flexibility, and also allows the user to be slowly weaned off of use of the orthotic device as the user's condition improves. The present invention thus permits multiple levels of support from a single brace by providing secondary body panels 16 that are configured to be removably attached to the primary body panel. The secondary body panels may be attached to provide added support and removed to provide less support.

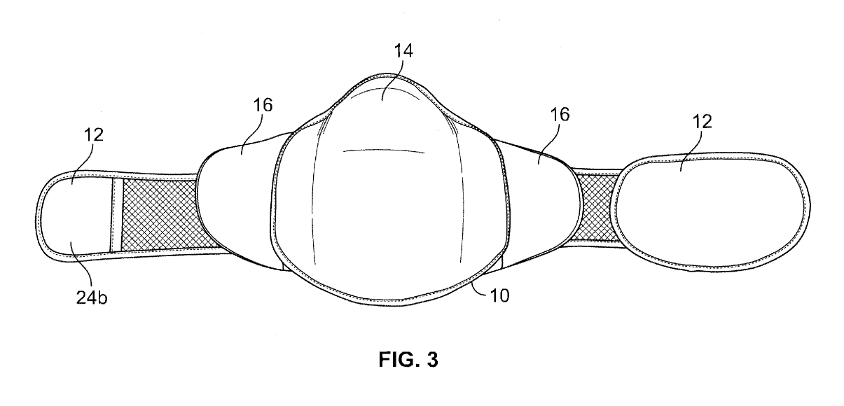

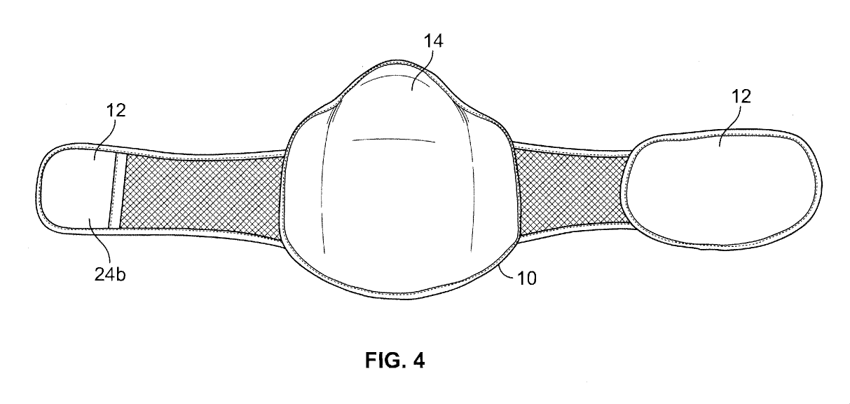

FIGS. 3 and 4 show front views of the orthotic device 10. FIG. 3 is a front view of the orthotic device with the left and right secondary body panels 16 attached to the primary body panel 14. The secondary body panels are configured to be releasably attached to the primary body panel and provide additional support to the user. FIG. 4 is a front view of the orthotic device with the secondary body panels removed. In this mode, the secondary body panels are no longer attached to the primary body panel, thus providing less support to the user. This allows the user to slowly wean himself or herself off of use of the device.

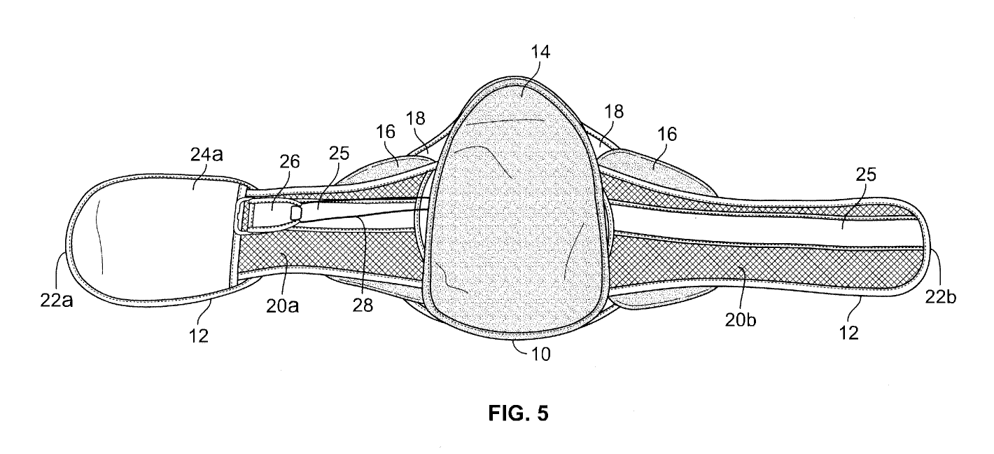

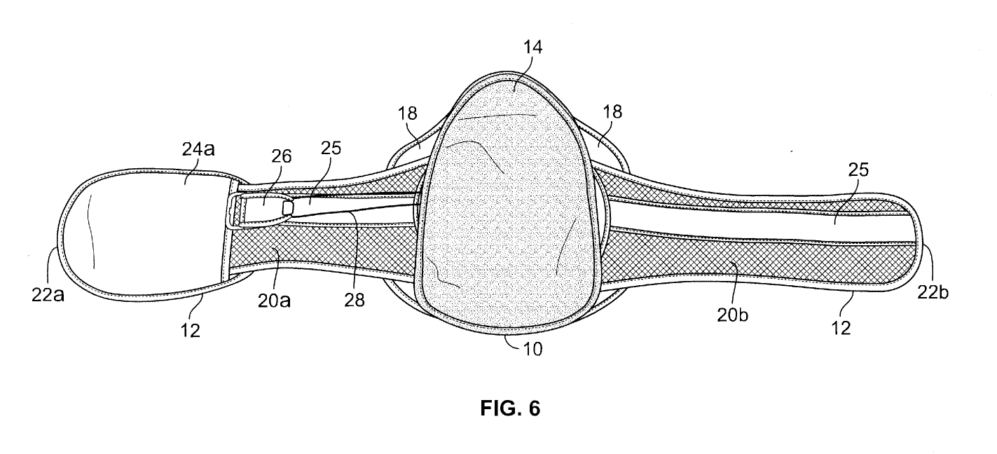

FIGS. 5 and 6 show rear views of the orthotic device 10. Like FIG. 3, FIG. 5 depicts the orthotic device with the left and right secondary body panels 16 removably attached to the primary body panel 14. In this mode, when the user straps the body brace 12 around his or her torso, the user's back will be supported by the primary body panel as well as the two secondary body panels, which provide additional support to the support provided by the primary body panel. FIG. 6 is a rear view of the orthotic device with the secondary body panels removed. In this mode, when the user straps the body brace around his or her torso, the user's back will be supported by only the primary body panel.

In one embodiment, the secondary body panels 16 are configured to be releasably attached to the primary body panel 14 through an attachment means 18. In a further embodiment, the attachment means is carried out using a hook-and-loop fastener. In this embodiment, at least a portion of the rear side of the primary body panel is covered with hook-and-loop fastener material. At least a portion of the front side of each of the secondary body panels is covered with complementary hook-and-loop fastener material, so that when the attachment surface of the secondary body panels comes into contact with the hook-and-loop fastener material on the primary body panel, the secondary body panels are removably attached to the primary body panel. Alternatively, buttons, snap fasteners, or similar fasteners may be employed to releasably secure the secondary body panels to the primary body panel.

In a further embodiment, the attachment means 18 permits each of the secondary body panels 16 to be attached to the primary body panel 14 in a variety of positions depending upon the needs of the user. For example, the user may place the secondary body panels so that there is greater or lesser horizontal overlap with the primary body panel. Similarly, the user may place the secondary body panel higher or lower on the primary body panel. In an embodiment using hook-and-loop fastener material, so long as the complementary sections of hook-and-loop fastener material on the attachment means 18 and secondary body panels 16 are in contact, then the secondary body panels will be removably attached to the primary body panel. This flexibility in placing the secondary body panels with respect to the primary body panel allows the user to place the secondary body panels in a variety of positions so that the user may customize the amount of support and comfort provided by orthotic device 10.

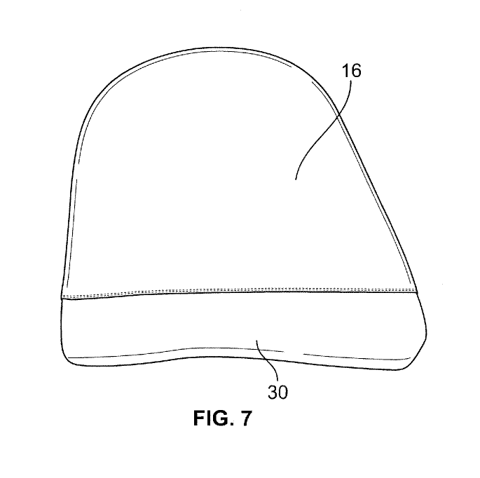

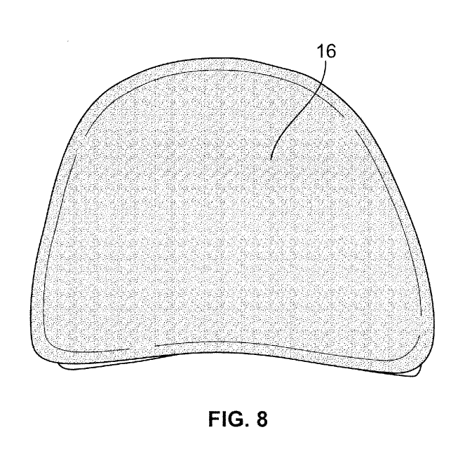

An embodiment of a left secondary body panel 16 is shown in FIGS. 7 and 8. It will be understood that the right secondary body panel may be a mirror image of the left secondary body panel.

FIG. 7 shows a front view of the left secondary body panel 16. The secondary body panel comprises a semi-rigid material that can provide added support to a portion of the user's body. In this particular embodiment, the secondary body panel is partially covered with hook-and-loop fastener material 30, so that when the hook-and loop-fastener material comes into contact with the attachment means 18 on the primary body panel 14, which comprises complementary sections of hook-and-loop fastener material, the secondary body panel will be removably attached to primary body panel.

With reference again to FIGS. 5 and 6, the body brace 12 may include two body brace segments 20a and 20b. On opposite overlapping sides of the opposite distal or free ends 22a and 22b of each body brace segment are provided complementary fastener or fastening means 24a and 24b for securing the two free ends together after the brace is wrapped around the patient's torso. Fastening means 24b is on the reverse side of distal end 22b and is depicted in FIGS. 3 and 4. Although buttons, snap fasteners, or similar fasteners may be employed to secure the free ends of the body brace together, a preferred embodiment uses complementary sections of hook-and-loop fastener fabric mounted on the opposite overlapping sides of the opposite body brace segments proximate at least the free ends thereof. As shown in FIGS. 4 and 5, the complementary portions of hook-and-loop fabric are represented by numerals 24a and 24b.

In addition to the complementary portions of hook-and-loop material at the free ends 22a and 22b of the body brace segments 20a and 20b, horizontal strips 25 of hook-and-loop material are arranged on the exterior surface of each body brace segment to allow for removably securing a handle element 26 to one of the body brace segments. The handle element is attached to the end of a cord 28 and used in the tension adjusters of a closure system for the orthotic device 10. The other end of the cord 28 is connected to a set of pulleys, which are configured in such a way that pulling the cord 28 will pull the body brace segments 20a and 20b together and tighten the body brace 12 around the user's torso. Such a pulley system is disclosed in greater detail in U.S. Pat. No. 6,213,968 to Heinz et al. or U.S. Pat. No. 35,940 to Heinz et al., which are incorporated herein by reference. The handle element 26 may include an easily graspable member, such as a cloth tab, loop, ring or bail. The handle 26 may also include a means of detachably securing the handle to a body brace segment after adjustment has been made or the device has been removed from the wearer. Such a means of securing could include a series of clasps, a buckle and strap, or a hook-and-loop arrangement. In the closure system shown in FIG. 5, a piece of hook-and-loop fabric is affixed to the underside of the handle element 26. After adjustment has been made and the appropriate tension has been established in the cord 28, the handle 26 may be releasably secured to a segment 20a or 20b by placing the hook-and-loop fabric on the handle 26 in contact with a portion of the complementary hook-and-loop material 25a or 25b on the body brace segment 20a or 20b.

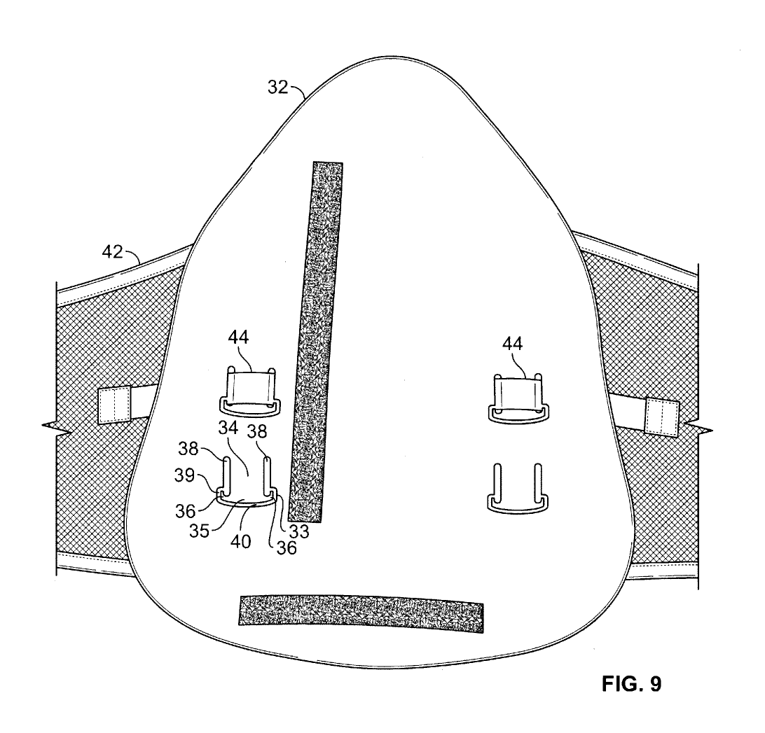

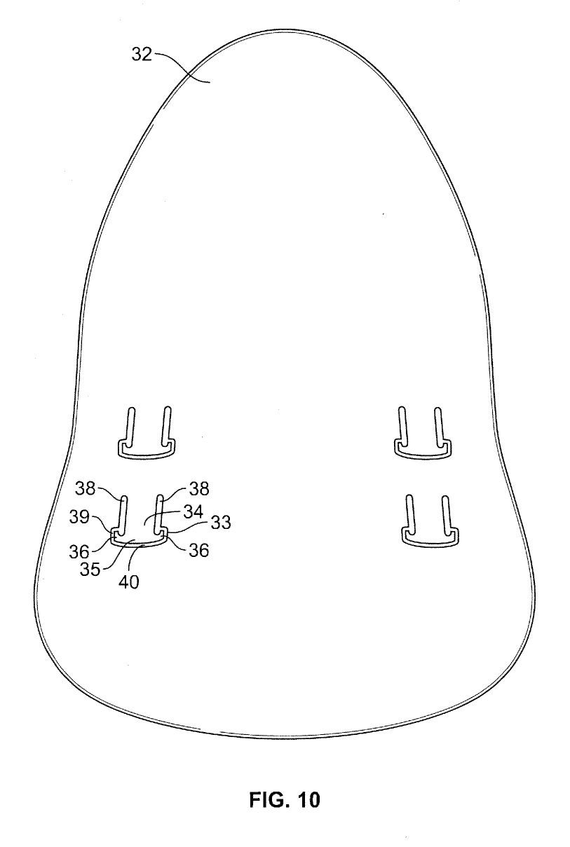

With reference to FIGS. 9 and 10, there is shown an embodiment of a primary body panel 32 that may be positioned on a rear portion of a body brace 42. The primary body panel 32 comprises four generally U-shaped apertures 33 for removably attaching the primary body panel to the rear portion of the body brace using straps, such as the straps 44 shown in FIG. 9. Each of the apertures extends about a single, generally flat retaining post 34 formed in the primary body panel 32. Each of the retaining posts comprises a flanged bottom portion 35 having a rounded bottom edge. At left and right ends, respectively, of the flanged bottom portion 35, two prongs 36 extend in an upward direction generally parallel with the retaining post.

Each of the apertures 33 comprises two slotted portions 38, two side portions 39, and a rounded bottom portion 40. Each slotted portion 38 borders one of the retaining posts 34 at a left or right edge thereof, and extends in a vertical direction generally parallel with the retaining post. Each side portion 39 extends from one of the slotted portions 38 about one of the prongs 36 to the rounded bottom portion 40. The rounded bottom portion 40 borders the rounded bottom edge of one of the flanged bottom portions 35.

To attach the primary body panel 32 to the rear portion of the body brace 42 using straps, such as the straps 44 shown in FIG. 9, a portion of a strap is slid through the rounded bottom portion 40 of one of the apertures 33, through the two side portions 39, and then into the two slotted portions 38. The strap presses against the retaining post 34 to retain the primary body panel in position with respect to the rear portion of the body brace. The two prongs 36 help retain the strap in position within the two slotted portions 38.

The primary body panel 32 comprises four apertures 33 (an upper set of two apertures and a lower set of two apertures). To attach the primary body panel 32 to the rear portion of the body brace 42, only the upper set of apertures or the lower set of apertures needs to be used. If the upper set of apertures is used, then the primary body panel 32 is positioned lower on the body brace, and thus lower on a patient's back. If the lower set of apertures is used, then the primary body panel 32 is positioned higher on the body brace, and thus higher on a patient's back. By providing two sets of apertures, the height of the primary body panel 32 can be adjusted with respect to the body brace. More or few sets of apertures may be provided.

Although the invention has been disclosed with reference only to the presently preferred embodiments, those of ordinary skill in the art will appreciate that various modifications can be made without departing from the invention. Accordingly, the invention is defined only by the following claims.

What is claimed is:

1. An orthotic device comprising:
a body brace configured to be worn around a portion of a human body;
a primary body panel positioned on a rear portion of the body brace; and
a secondary body panel configured to be releasably attached to the primary body panel for providing an adjustable level of support to the portion of the human body.

2. The orthotic device of claim 1, wherein:
the primary body panel has hook-and-loop fastener material; and
the secondary body panel has complementary hook-and-loop fastener material for releasably attaching the secondary body panel to the primary body panel.

3. The orthotic device of claim 1, wherein the secondary body panel is configured to be releasably attached to the primary body panel in a plurality of positions.

4. The orthotic device of claim 1, wherein the secondary body panel comprises a semi-rigid material.

5. The orthotic device of claim 1, wherein:
the body brace comprises
two body brace segments,
a pulley system mounted on the two body brace segments, and
a cord wrapped around the pulley system; and
the body brace may be adjusted by pulling the cord.

6. The orthotic device of claim 1, wherein the primary body panel comprises a plurality of apertures and a plurality of retaining posts for removably attaching the primary body panel to the rear portion of the body brace; and
wherein each of the apertures extends about one of the retaining posts.

7. The orthotic device of claim 6, wherein the plurality of apertures comprises an upper set of two apertures and a lower set of two apertures.

8. The orthotic device of claim 6, wherein each of the apertures comprises two slotted portions for receiving a strap.

9. The orthotic device of claim 6, wherein each of the retaining posts comprises a flanged bottom portion.

10. The orthotic device of claim 10, wherein the flanged bottom portion comprises two prongs extending from opposite ends of the flanged bottom portion.

11. A method for receiving adjustable levels of support using an orthotic device, the method comprising the steps of
obtaining an orthotic device comprising
a body brace configured to be worn around a portion of a human body,
a primary body panel positioned on a rear portion of the body brace, and
a secondary body panel configured to be releasably attached to the primary body panel;
wearing the orthotic device with the secondary body panel attached to the primary body panel;
doffing the orthotic device;
removing the secondary body panel from the primary body panel; and
wearing the orthotic device without the secondary body panel attached to the primary body panel.

12. The method of claim 11, wherein:
the primary body panel has hook-and-loop fastener material; and
the secondary body panel has complementary hook-and-loop fastener material for releasably attaching the secondary body panel to the primary body panel.

13. The method of claim 11 wherein the secondary body panel is configured to be releasably attached to the primary body panel in a plurality of positions.

14. The method of claim 11, wherein the secondary body panel comprises a semi-rigid material.

15. The method of claim 11, wherein:
the body brace comprises
two body brace segments,
a pulley system mounted on the two body brace segments, and
a cord wrapped around the pulley system; and
the body brace may be adjusted by pulling the cord.

16. The method of claim 15 wherein the steps of wearing the orthotic device include the step of adjusting the body brace by pulling the cord.

17. An orthotic device comprising:
a body brace configured to be worn around a portion of a human body; and
a body panel positioned on a rear portion of the body brace;
wherein the body panel comprises a plurality of apertures and a plurality of retaining posts for removably attaching the body panel to the rear portion of the body brace; and
wherein each of the apertures extends about one of the retaining posts.

18. The orthotic device of claim 17, wherein the plurality of apertures comprises an upper set of two apertures and a lower set of two apertures.

19. The orthotic device of claim 17, wherein each of the apertures comprises two slotted portions for receiving a strap.

20. The orthotic device of claim 17, wherein each of the retaining posts comprises a flanged bottom portion.

21. The orthotic device of claim 20, wherein the flanged bottom portion comprises two prongs extending from opposite ends of the flanged bottom portion.
